(19) United States
(12) Patent Application Publication
(10) Pub. No.: US 2025/0064573 A1
(43) Pub. Date: Feb. 27, 2025

(54) Vascular Implant

(71) Applicant: BARD PERIPHERAL VASCULAR, INC., Franklin Lakes, NJ (US)

(72) Inventors: Matt Casiraro, Tempe, AZ (US); Alexander Lastovich, Gilbert, AZ (US)

(73) Assignee: BARD PERIPHERAL VASCULAR, INC., Franklin Lakes, NJ (US)

(21) Appl. No.: 18/724,270

(22) PCT Filed: Dec. 30, 2021
(86) PCT No.: PCT/US2021/073178

§ 371 Date: Jun. 26, 2024

(51) Int. Cl. A61F 2/01

(52) U.S. Cl. CPC A61F 2/0105; A61F 2002/016; A61F 2220/0016; A61F 2240/001

(57) ABSTRACT

A vascular implant body having a hub or ring and having a central opening, a central longitudinal axis and an outer ring periphery. Multiple appendages each extend radially from the hub or ring. Engagement anchors on the appendages each extend outwardly toward the distal end portion of an adjacent appendage.

### CROSS-REFERENCE TO RELATED APPLICATIONS

N/A

### STATEMENT REGARDING FEDERALLY SPONSORED RESEARCH OR DEVELOPMENT

Not applicable

### REFERENCE TO A “MICROFICHE APPENDIX”

Not applicable

### BACKGROUND OF THE INVENTION

### 1. Field of the Invention

The present invention relates to bioresorbable vascular implants such as filters (e.g., vena cava filters), occlusion devices, stents and convertible stents. In one embodiment, the implant body has a centrally located ring and radially extending appendages connected to and extending away from the anchor ring. Torsional vessel engagement hooks extend from the central ring. In one embodiment, the vascular implant bioresorbs into a patient's vascular system (e.g., inferior vena cava, iliofemoral vein, ovarian veins, splenic artery, uterine artery, hepatic artery or other vein/artery vessel). For the inferior vena cava and iliofemoral vein, the implant resorbs after transient risk of pulmonary embolism (PE) has subsided. The other mentioned veins and arteries are receptive of an occlusion device implant of the present invention. The entire implant structure could be made of bioresorbable material so that no implant or implant remnant/element would ultimately be left behind as the entire implant would resorb into vascular tissue. In one embodiment, the implant is specifically configured and 3D printable having a ring portion and radially extending torsional vessel engagement anchors or hooks.

### 2. General Background of the Invention

Vascular implants include various devices that are placed at a selected locale in a patient's blood vessel. One example is a vena cava filter. Another example is an occlusion device. Various patents have issued for vascular implants. Patents have also issued that relate in general to 3D printing of implants. Examples are listed in the following Table 1. Each patent listed in Table 1 is hereby incorporated herein by reference

TABLE 1

| PATENT OR | | |
|---|---|---|
| PUBLICATION | | ISSUE DATE |
| NO. | TITLE | (DD/MM/YYYY) |
| 8,092,484 | EMBOLUS BLOOD CLOT FILTER WITH | 10 Jan. 2012 |
| | POST DELIVERY ACTUATION | |
| 8,092,485 | RECOVERABLE INFERIOR VENA CAVA | 10 Jan. 2012 |
| | FILTER | |
| 8,317,818 | REMOVABLE BLOOD CLOT FILTER | 27 Nov. 2012 |
| | WITH EDGE FOR CUTTING THROUGH | |
| | THE ENDOTHELIUM | |
| 8,420,113 | BIODEGRADABLE MEDICAL DEVICES | 16 Apr. 2013 |
| | WITH ENHANCED MECHANICAL | |
| | STRENGTH AND PHARMACOLOGICAL | |
| | FUNCTIONS | |
| 8,518,072 | JUGULAR FEMORAL VENA CAVA | 27 Aug. 2013 |
| | FILTER SYSTEM | |
| 8,562,638 | EMBOLUS BLOOD CLOT FILTER WITH | 22 Oct. 2013 |
| | FLOATING FILTER BASKET | |
| 8,734,479 | EMBOLUS BLOOD CLOT FILTER | 27 May 2014 |
| | DELIVERY SYSTEM | |
| 8,777,975 | EMBOLUS BLOOD CLOT FILTER WITH | 15 Jul. 2014 |
| | BIO-RESORBABLE COATED FILTER | |
| | MEMBERS | |
| 8,795,351 | MIGRATION RESISTANT EMBOLIC | 5 Aug. 2014 |
| | FILTER | |
| 8,870,943 | STENT STRUCTURE FOR | 28 Oct. 2014 |
| | IMPLANTATBLE MEDICAL DEVICE | |
| 8,992,562 | FILTER DELIVERY SYSTEM | 31 Mar. 2015 |
| 9,220,588 | SYSTEMS, METHODS AND DEVICE FOR | 29 Dec. 2015 |
| | EMBOLIC PROTECTION | |
| 9,393,095 | JUGULAR FEMORAL VENA CAVA | 19 Jul. 2016 |
| | FILTER SYSTEM | |
| 9,421,081 | EMBOLUS BLOOD CLOT FILTER | 23 Aug. 2016 |
| | DELIVERY SYSTEM | |
| 9,445,895 | INTRACARDIAC CAGE AND METHOD OF | 20 Sep. 2016 |
| | DELIVERING SAME | |
| 9,456,888 | REVERSIBLE VASCULAR FILTER | 4 Oct. 2016 |
| | DEVICES AND METHODS FOR USING | |
| | SAME | |
| 9,468,513 | EMBOLUS BLOOD CLOT FILTER WITH | 18 Oct. 2016 |
| | BIO-RESORBABLE COATED FILTER | |
| | MEMBERS | |
| 9,561,094 | DEVICES AND METHODS FOR TREATING | 7 Feb. 2017 |
| | VENOUS DISEASES | |
| 9,597,435 | MEDICAL DEVICES HAVING A | 21 Mar. 2017 |
| | BIORESORBABLE COATING LAYER | |
| | WITH A PRE-DETERMINED PATTERN | |
| | FOR FRAGMENTATION | |
| 9,693,851 | FILTER DELIVERY SYSTEM | 4 Jul. 2017 |
| 9,730,781 | EMBOLUS BLOOD CLOT FILTER | 15 Aug. 2017 |
| | REMOVAL SYSTEM AND METHOD | |
| 9,949,816 | IVC FILTER RETRIEVAL SYSTEMS WITH | 24 Apr. 2018 |
| | MULTIPLE CAPTURE MODES | |
| 9,980,804 | VENA CAVA FILTER WITH FILAMENT | 29 May 2018 |
| 10,105,206 | INFERIOR VENA CAVA FILTER WITH | 23 Oct. 2018 |
| | STABILITY FEATURES | |
| 10,188,496 | VENA CAVA FILTER FORMED FROM A | 29 Jan. 2019 |
| | SHEET | |
| 10,188,498 | EMBOLUS BLOOD CLOT FILTER | 29 Jan. 2019 |
| | DELIVERY SYSTEM | |
| 10,226,322 | JUGULAR FEMORAL VENA CAVA | 12 Mar. 2019 |
| | FILTER SYSTEM | |
| 10,258,454 | VISUAL STABILIZER ON ANCHOR LEGS | 16 Apr. 2019 |
| | OF VENA CAVA FILTER | |
| 10,279,078 | CROSSLINKABLE 3D PRINTED | 7 May 2019 |
| | BIOMATERIAL-BASED IMPLANTS AND | |
| | METHODS OF MANUFACTURE THEREOF | |
| 10,299,906 | EMBOLUS BLOOD CLOT FILTER | 28 May 2019 |
| | UTILIZABLE WITH SINGLE DELIVERY | |
| | SYSTEM OR A SINGLE RETRIEVAL | |
| | SYSTEM IN ONE OF A FEMORAL OR | |
| | JUGULAR ACCESS | |
| 10,342,654 | IVC FILTER WITH TRANSLATING HOOKS | 9 Jul. 2019 |
| 10,368,972 | EMBOLUS BLOOD CLOT FILTER WITH | 6 Aug. 2019 |
| | BIO-RESORBABLE COATED FILTER | |
| | MEMBERS | |
| 10,390,925 | MIGRATION RESISTANT EMBOLIC | 27 Aug. 2019 |
| | FILTER | |
| 10,441,689 | METHODS AND DEVICES FOR THREE- | 15 Oct. 2019 |
| | DIMENSIONAL PRINTING OR ADDITIVE | |
| | MANUFACTURING OF BIOACTIVE | |
| | MEDICAL DEVICES | |
| 10,470,865 | VASCULAR FILTER DEVICE | 12 Nov. 2019 |
| 10,492,898 | EMBOLUS BLOOD CLOT FILTER AND | 3 Dec. 2019 |
| | DELIVERY SYSTEM | |
| 10,512,531 | FILTER DELIVERY SYSTEM | 24 Dec. 2019 |
| 10,531,942 | ABSORBABLE VASCULAR FILTER | 14 Jan. 2020 |
| 10,579,755 | METHOD FOR 3-D PRINTING A CUSTOM | 3 Mar. 2020 |
| | BONE GRAFT | |
| 10,624,731 | VASCULAR FILTER SYSTEM | 21 Apr. 2020 |
| 10,729,527 | REMOVABLE EMBOLUS BLOOD CLOT | 4 Aug. 2020 |
| | FILTER | |
| 10,813,738 | TUBULAR FILTER | 27 Oct. 2020 |
| 10,842,608 | VENA CAVA FILTER WITH FILAMENT | 24 Nov. 2020 |
| 2007/0064731 | TRANSMISSION APPARATUS WITH | 22 Mar. 2007 |
| | FUNCTION OF MULTI-STEP BANDWIDTH | |
| | ASSIGNMENT TO OTHER | |
| | COMMUNICATION APPARATUSES | |
| 2010/0074934 | MEDICAL IMPLANTS WITH A | 25 Mar. 2010 |
| | COMBINATION OF COMPOUNDS | |
| 2016/0166371 | ENDOLUMINAL FILTER DESIGN | 16 Jun. 2016 |
| | VARIATIONS | |
| 2016/0175085 | ENHANCED FLUOROGENIC | 23 Jun. 2016 |
| | ENDOLUMINAL FILTER STRUCTURE | |
| 2017/0105830 | BIODEGRADABLE VASCULAR FILTER | 20 Apr. 2017 |
| 2017/0218228 | THREE DIMENSIONAL PRINTING OF BIO- | 3 Aug. 2017 |
| | INK COMPOSITIONS | |
| 2017/0249440 | 3D PRINTING SURGICAL REPAIR | 31 Aug. 2017 |
| | SYSTEMS | |
| 2017/0340429 | VASCULAR FILTER SYSTEM | 30 Nov. 2017 |
| 2018/0168811 | NOVEL BIODEGRADABLE AND NON- | 21 Jun. 2018 |
| | BIODEGRADABLE 3D PRINTED | |
| | IMPLANTS AS A DRUG DELIVERY | |
| | SYSTEM | |
| 2018/0296343 | 3-D PRINTING OF POROUS IMPLANTS | 18 Oct. 2018 |
| 2018/0303616 | 3-D PRINTING OF BONE GRAFTS | 25 Oct. 2018 |
| 2018/0311028 | VENA CAVA FILTER WITH FILAMENT | 1 Nov. 2018 |
| 2019/0110880 | MEDICAL DEVICES AND ANCHORS | 18 Apr. 2020 |
| | THREFOR | |
| 2020/0001540 | ADDITIVE MANUFACTURING ON | 2 Jan. 2020 |
| | UNCONSTRAINED FREEFORM | |
| | SURFACES | |
| 2020/0197150 | VASCULAR FILTER SYSTEM | 25 Jun. 2020 |
| WO2007064731 | HELICAL VENA CAVA FILTER | 7 Jun. 2007 |
| WO2011079287 | REVERSIBLE VASCULAR FILTER | 30 Jun. 2011 |
| | DEVICES AND METHODS FOR USING | |
| | SAME | |
| WO2016154148 | ARTIFICIAL TYMPANIC MEMBRANE | 29 Sep. 2016 |
| | DEVICES AND USES | |
| WO2018117907 | SHAPE MEMORY POLYMER COMPOSITE | 28 Jun. 2018 |
| | FOR 3D PRINTING OF MEDICAL ITEMS | |
| WO2018218085 | THREE-DIMENSIONAL PRINTED | 29 Nov. 2018 |
| | ORGANS, DEVICES, AND MATRICES | |
| WO2019178086 | ELECTROHYDRODYNAMIC BIOPRINTER | 19 Sep. 2019 |
| | SYSTEM AND METHOD | |
| WO2020123945 | FABRIC MATERIAL FOR MEDICAL | 18 Jun. 2020 |
| | DEVICES | |
| EP2363156 | METHOD OF FABRICATING | 07 Sep. 2011 |
| | BIODEGRADABLE MEDICAL DEVICES | |
| | WITH ENHANCED MECHANICAL | |
| | STRENGTH AND PHARMACOLGOICAL | |
| | FUNCTIONS | |

### BRIEF SUMMARY OF THE INVENTION

The present invention provides a vascular implant body preferably including a hub or ring having a central opening, a central longitudinal axis and an outer ring periphery.

In one or more embodiments, multiple appendages each preferably extend radially from the hub or ring.

In one or more embodiments, engagement anchors on the appendages each preferably extend outwardly toward the distal end portion of an adjacent appendage.

In one or more embodiments, the engagement anchors preferably prevent rotation of the implant body in a selected rotational direction.

In one or more embodiments, each anchor is a hook that preferably extends along a circular path toward the anchor of an adjacent appendage.

In one or more embodiments, each appendage preferably has a bend.

In one or more embodiments, each appendage preferably has a first straight section that connects to the hub or ring and a second straight section that connects to the anchor.

In one or more embodiments, the selected rotational direction is clockwise or counterclockwise relative to the central longitudinal direction.

In one or more embodiments, each of the anchors preferably has a pointed projection.

In one or more embodiments, each anchor preferably includes a sharp pointed portion.

In one or more embodiments, each anchor preferably includes a hook.

In one or more embodiments, the hub or ring occupies a plane and each appendage preferably forms an acute angle with the plane.

In one or more embodiments, an implant body includes a hub or ring having a central opening, a central longitudinal axis, and an outer ring periphery.

In one or more embodiments, the implant body preferably includes multiple appendages that each connect with the hub or ring.

In one or more embodiments, engagement anchors on the implant body resist torsion, each anchor preferably extends generally circumferentially toward an anchor so that all of the anchors resist rotation of the implant body in a selected rotational direction.

In one or more embodiments, each anchor preferably is a hook that extends along a circular path toward the anchor of an adjacent appendage.

In one or more embodiments, each appendage preferably has a bend portion.

In one or more embodiments, each of the anchors preferably is a pointed projection.

In one or more embodiments, an implant body preferably includes a hub or ring that has a central opening, a central longitudinal axis, and an outer ring periphery.

In one or more embodiments, multiple appendages preferably each connect with the hub or ring.

In one or more embodiments, multiple engagement anchors are preferably on the implant body, each having a pointed or sharp tip that extends toward another, adjacent engagement anchor.

In one or more embodiments, the engagement anchors are positioned to engage a patient's vascular tissue so that rotation of the implant body is resisted in a selected rotational direction.

In one or more embodiments, each engagement anchor preferably forms a hook or spike.

In one or more embodiments, each engagement anchor preferably has a curvature.

### BRIEF DESCRIPTION OF THE SEVERAL VIEWS OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is a top view of a preferred embodiment of the apparatus of the present invention;

FIG. 2 is a side view of a preferred embodiment of the apparatus of the present invention; and

FIG. 3 is a perspective view of a preferred embodiment of the apparatus of the present invention.

### DETAILED DESCRIPTION OF THE INVENTION

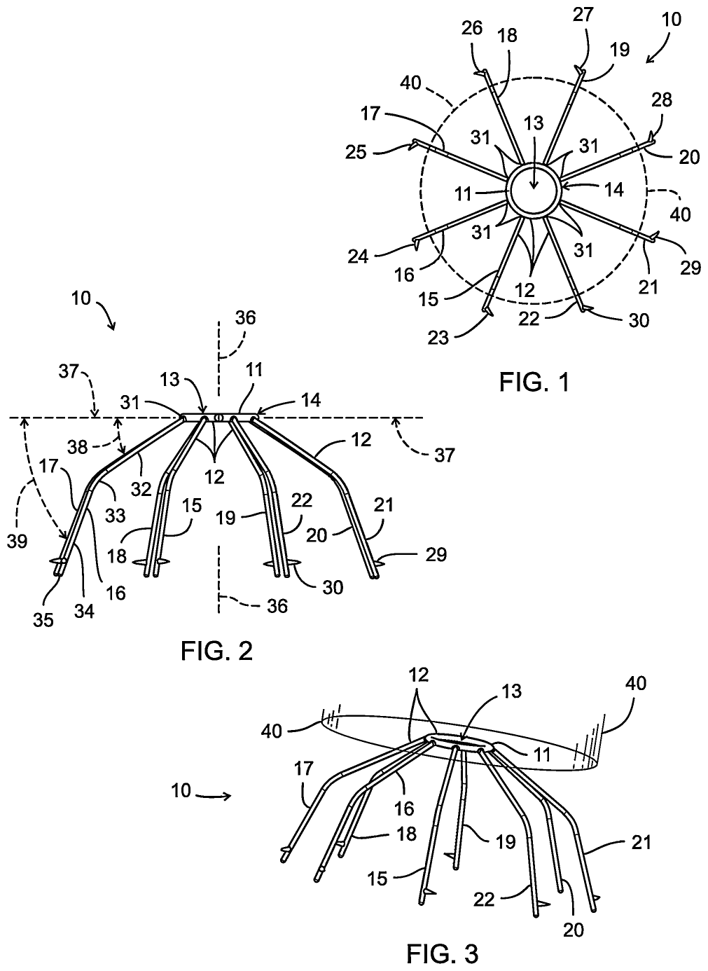

FIGS. 1-3 show a preferred embodiment of the apparatus of the present invention, designated generally by the numeral 10. Implant 10 includes a body 12 that comprises a hub, ring or head 11 and attached appendages 15-22, which may also be referred to as legs or arms. Hub, ring or head 11 can have a central opening 13 and a hub, ring or head periphery 14. The central opening 13 can reduce the fluid resistivity of the body 12.

Appendages 15-22 extend both radially and axially from the hub, ring or head 11. Each of the legs 14-19 has a proximal end next to the hub, ring or head 11 and a distal end with an anchor for engaging a vessel. In this embodiment, the distal end forms a free-end of the respective appendage 14-19 whereas the proximal end is connected to the hub, ring or head 11.

The implant is to be placed in the vessel with the head downstream, in a direction of flow through the vessel, from the distal ends. The distal ends thus form a bottom of the body 12. The body 12 is open at the bottom, and seen in radial direction, the bottom is wider than the head. In this embodiment the distal ends form the bottom of the body 12. The body 12 has a frusto-conical shape with an open base, with the head 11 forming the closed top and the bottom formed by the distal ends forming the base of the conus. The terms “closed” and “open” are with reference to particles to be filtered or retained by the implant, which can enter the conus at the base but not leave via the top whereas bodily fluid can pass both at the base and the top. The appendages may form an open cell filter or a closed cell filter, for instance.

When the vascular implant is placed in the vessel, bodily fluid, e.g. blood, transported through the vessel will flow, substantially in the longitudinal direction of the body 12, in the direction from the distal ends of the appendages 15-22 towards the hub, ring or head 11 and pass through the body 10 to be filtered. The fluid will flow through the space between the appendages 15-22, as well as in this embodiment through the central opening 13 of the hub, ring or head 11. Clots that cannot pass through this space and, if present, through the central opening 13, are blocked from passing through.

The appendages 15-22 are provided with engagement anchors 23-30. One or more, in this example each, of said anchors 23-30 may extend outwardly, towards the distal end portion of an adjacent appendage. When the implant 10 is placed in a vessel, the engagement anchors project into the wall of the vessel and prevent rotation of the implant body in rotational direction opposite to the tangential direction in which the anchors project. The risks of complications caused by the implant can thereby be reduced. In this example, the anchors 23-30 project outwards in a radial-tangential direction from the respective appendage 15-22, and thus anchor the implant both in the rotational direction and the axial direction. By having the device rotated in to place it is less susceptible to being dislodged along the axis of flow and thus reduces the potential for unwanted migration or dislodgement when compared to something that is in-line with flow and could be moved along the axis and may have a higher chance of having the anchor features removed from the vessel wall.

In this embodiment, hub, ring or head 11 can occupy plane 37 (see FIG. 2). Each appendage 15-22 has one or more appendage sections that form an acute angle with plane 37. Each appendage can include an upper or proximal section 32, middle section 33 and a lower or distal section 34. The acute angle can differ between sections, and for example can increase from the proximal end to the distal end of the appendage. In FIG. 2, numeral 38 represents the acute angle between upper appendage section 32 and plane 37. Reference numeral 39 represents the acute angle between lower appendage section 34 and plane 37. Angle 38 can be between about 19 and 49 degrees. Angle 39 can be between about 55 and 85 degrees.

Each appendage 15-22 has a distal end 35 (see FIG. 2). In this embodiment, each appendage 15-22 is attached to ring, head or hub 11 periphery 14 with the proximal end, with attachment 31. However, the head, hub or ring 11 and appendages 15-22 can be an integral or one piece construction such as printed with a 3D printer. For inferior vena cava pulmonary embolism protection, implant 10 can be about 15-30 mm in diameter. For iliofemoral pulmonary embolism prevention, implant 10 can be between about 6 and 20 mm in diameter. For other smaller vessels for occlusion, the diameter of implant 10 can be about 2-8 mm. Implant 10 can be about 5-50 mm in length.

Implant body 12 can have a central longitudinal axis 36. In one embodiment, spikes, projections, anchors or hooks 23-30 are provided that prevent rotation of implant body 12 around this axis 36 in a selected rotational direction after implant body 12 is placed in a patient's selected vascular tissue 40. In FIG. 1, the spikes, projections, barbs, hooks or anchors 23-30 prevent counterclockwise rotation, as each barb, hook, spike, projection or anchor 23-30 extends from a distal end portion or distal end 35 of an appendage 15-22 toward another, adjacent appendage 15-22 and its anchor 23-30. For example, the barb, hook, spike, projection or anchor 23 on appendage 15 extends toward the barb, hook, spike, projection or anchor 30 on appendage 22. Similarly, the barb, hook, spike, projection or anchor 30 on appendage 22 extends toward the barb, hook, spike, projection or anchor 29 on appendage 21. In like fashion, anchor 29 extends toward anchor 28, anchor 28 extends toward anchor 27, anchor 27 extends toward anchor 26, anchor 26 extends toward anchor 25, anchor 25 extends toward anchor 24 and anchor 24 extends toward anchor 23. Each anchor can be curved, bent or straight. In this embodiment, the anchors allow rotation in the direction opposite to the selected rotational direction, and in this example clock-wise direction is allowed.

Each appendage 15-22 can have upper or proximal section 32. Section 32 can be a straight section. Each appendage 15-22 can have a distal end 35. Each anchor can form an angle with distal or lower section 34 of appendage. Implant body 12 can be of a material that can be 3D printed. Implant body 12 can be printed with a 3D printer. Implant body 12 can be of a resorbable material such as a bioresorbable polymer. When of a bioresorbable material, the bioresorption starts as soon as the implant is exposed to the blood and like many of the standard polymers degrades over time via polymer breakdown and absorption of the byproducts. This bioresorption can be tuned as needed to ensure the implant maintains structural filtering integrity until transient risk of pulmonary embolism has subsided. In one embodiment, implant body 12 is of a material that is too soft to be machined, such as a material having a durable reading below 20 Shore D.

The implant 10 could be deployed with either a femoral or jugular approach. Such a deployment could employ a pusher or pusher apparatus/mechanism such as one specified in one or more of the patents listed in Table 1. An example is U.S. Pat. No. 8,518,072 naming Jonathan Miller as inventor and assigned to C.R. Bard, Inc. Implant 10 could also be a balloon-mounted implant that is then expanded with balloon dilation, as seen, for example, in balloon expandable stents.

The vascular implant can thus be characterized by comprising an implant body that includes a hub or ring, multiple appendages that each extend radially from the hub or ring, each appendage having a proximal section and a distal end portion; and engagement anchors on the appendages that each extend outwardly, towards the distal end portion of an adjacent appendage. The engagement anchors prevent rotation of the implant body in a selected rotational direction. Without limitation, the implant may further be characterized by one or more of the following statements.

Statement 1: A vascular implant, comprising:
a) an implant body that includes:
   a hub or ring having a central opening,
   a central longitudinal axis, and
   an outer ring periphery;
b) multiple appendages that each extend radially from the hub or ring, each appendage having a proximal section and a distal end portion;
c) engagement anchors on the appendages that each extend outwardly toward the distal end portion of an adjacent appendage; and
d) wherein the engagement anchors prevent rotation of the implant body in a selected rotational direction.

Statement 2: The vascular implant of one or more of the preceding statements wherein each anchor is a hook that extends along a circular path toward the anchor of an adjacent appendage.

Statement 3: The vascular implant of one or more of the preceding statements wherein each appendage has a bend.

Statement 4: The vascular implant of one or more of the preceding statements wherein each appendage has a first straight section that connects to the hub or ring and a second straight section that connects to the anchor.

Statement 5: The vascular implant of one or more of the preceding statements wherein the selected rotational direction is clockwise or counterclockwise relative to the central longitudinal axis.

Statement 6: The vascular implant of one or more of the preceding statements wherein each of the anchors is a pointed projection.

Statement 7: The vascular implant of one or more of the preceding statements wherein each the anchor includes a sharp pointed portion.

Statement 8: The vascular implant of one or more of the preceding statements wherein each the anchor includes a hook.

Statement 9: The vascular implant of one or more of the preceding statements wherein the hub or ring occupies a plane and each appendage forms an acute angle with the plane.

Statement 10: A vascular implant, comprising:
a) an implant body that includes a hub or ring having a central opening, a central longitudinal axis and an outer ring periphery;
b) the implant body including multiple appendages that each connect with the hub or ring; and
c) engagement anchors on the implant body that resist torsion, each the anchor extending generally circumferentially toward an anchor so that all of the anchors resist rotation of the implant body in a selected rotational direction.

Statement 11: The vascular implant of statement 10 wherein each anchor is a hook that extends along a circular path toward the anchor of an adjacent appendage.

Statement 12: The vascular implant of statement 10 or 11 wherein each appendage has a bend portion.

Statement 13: The vascular implant of one or more of statements 10-12 wherein each appendage has a first straight section that connects to the hub or ring and a second straight section that connects to the anchor.

Statement 14: The vascular implant of one or more of statements 10-13 wherein each of the anchors is a pointed projection.

Statement 15: The vascular implant of one or more of statements 10-14 wherein each the anchor includes a sharp pointed portion.

Statement 16: The vascular implant of one or more of statements 10-15 wherein each the anchor includes a hook.

Statement 17: The vascular implant of one or more of statements 10-16 wherein the hub or ring occupies a plane and each appendage forms an acute angle with the plane.

Statement 18: A vascular implant, comprising:
a) an implant body that includes a hub or ring having a central opening, a central longitudinal axis and an outer ring periphery;
b) the implant body including multiple appendages that each connect with the hub or ring;
c) multiple engagement anchors on the implant body that each have a pointed or sharp tip that extends toward another, adjacent engagement anchor; and
d) wherein the engagement anchors are positioned to engage a patient's vascular tissue so that rotation of the implant body is resisted in a selected rotational direction.

Statement 19: The vascular implant of statement 18 wherein each engagement anchor forms a hook or spike.

Statement 20: The vascular implant of statement 18 or 19 wherein each engagement anchor has a curvature.

The following is a list of parts and materials suitable for use in the present invention.

### PARTS LIST

| Part Number | Description |
|---|---|
| 10 | vascular implant |
| 11 | hub, ring, head |
| 12 | implant body |
| 13 | central opening |
| 14 | hub, ring or head periphery |
| 15 | appendage |
| 16 | appendage |
| 17 | appendage |
| 18 | appendage |
| 19 | appendage |
| 20 | appendage |
| 21 | appendage |
| 22 | appendage |
| 23 | anchor, hook, spike, barb, sharp projection |
| 24 | anchor, hook, spike, barb, sharp projection |
| 25 | anchor, hook, spike, barb, sharp projection |
| 26 | anchor, hook, spike, barb, sharp projection |
| 27 | anchor, hook, spike, barb, sharp projection |
| 28 | anchor, hook, spike, barb, sharp projection |
| 29 | anchor, hook, spike, barb, sharp projection |
| 30 | anchor, hook, spike, barb, sharp projection |
| 31 | attachment |
| 32 | straight section/upper appendage |
| | section/proximal section |
| 33 | middle section/bend section |
| 34 | proximal section/straight section/lower |
| | appendage section/distal section |
| 35 | distal end |
| 36 | central longitudinal axis |
| 37 | plane |
| 38 | angle |
| 39 | angle |
| 40 | vessel/vascular tissue |

All measurements disclosed herein are at standard temperature and pressure, at sea level on Earth, unless indicated otherwise. All materials used or intended to be used in a human being are biocompatible, unless indicated otherwise.

The foregoing embodiments are presented by way of example only; the scope of the present invention is to be limited only by the following claims.

1. A vascular implant, comprising:
a) an implant body that includes:
a hub or ring having a central opening, said hub or ring defining a plane,
a hub central longitudinal axis, and
an outer hub or ring periphery;
b) multiple appendages that each extend radially from said hub or ring, each appendage having a proximal appendage section and a distal end appendage section;
c) wherein each said proximal appendage section forms an acute angle with said plane;
d) an engagement anchor on each said appendages;
e) each engagement anchor extending away from a said appendage and toward an adjacent engagement anchor along a generally circular path; and
f) wherein said engagement anchors prevent rotation of said implant body in a selected rotational direction.

2. The vascular implant of claim 1 wherein each engagement anchor is a hook that extends along a circular path toward the engagement anchor of an adjacent appendage.

3. The vascular implant of claim 1 wherein each appendage has a bend that connects each proximal appendage section to a said distal appendage section.

4. The vascular implant of claim 1 wherein each appendage proximal section is a first straight section that connects to the hub or ring and said distal appendage section is a second straight section that connects to a said engagement anchor.

5. The vascular implant of claim 1 wherein the selected rotational direction is clockwise or counterclockwise relative to said central longitudinal axis.

6. The vascular implant of claim 1 wherein each of said anchors is a pointed projection.

7. The vascular implant of claim 1 wherein each said anchor includes a sharp pointed portion.

8. The vascular implant of claim 1 wherein each said anchor includes a hook.

9. The vascular implant of claim 1 wherein said hub or ring occupies a plane and at least one section of each appendage forms an acute angle with said plane.

10. A vascular implant, comprising:
a) an implant body that includes a hub or ring having a central opening, a central longitudinal axis and an outer ring periphery;
b) said implant body including multiple appendages that each connect with said hub or ring at said outer periphery; and
c) engagement anchors on said implant body that resist torsion, each said anchor extending along a circular path toward another said engagement anchor so that all of the anchors resist rotation of the implant body in a selected rotational direction.

11. The vascular implant of claim 10 wherein each engagement anchor is a hook that extends along a circular path toward the engagement anchor of an adjacent appendage.

12. The vascular implant of claim 10 wherein each appendage has a bend portion.

13. The vascular implant of claim 10 wherein each appendage has a first straight section that connects to the hub or ring and a second straight section that connects to a said engagement anchor.

14. (canceled)

15. The vascular implant of claim 10 wherein each said anchor includes a sharp pointed portion.

16. The vascular implant of claim 10 wherein each said anchor includes a hook.

17. The vascular implant of claim 10 wherein said hub or ring occupies a plane and each appendage has a section that forms an acute angle with said plane.

18. A vascular implant, comprising:
a) an implant body that includes a hub or ring having a central opening, a central longitudinal axis and an outer ring periphery;
b) said implant body including multiple appendages that each connect with said hub or ring, each said appendage having a distal end portion;
c) engagement anchors on each said appendage distal end portion that each have a pointed or sharp tip that extends toward another, adjacent engagement anchor; and
d) wherein the engagement anchors are positioned to engage a patient's vascular tissue so that rotation of the implant body is resisted in a selected rotational direction.

19. The vascular implant of claim 18 wherein each engagement anchor forms a hook or spike.

20. The vascular implant of claim 18 wherein each engagement anchor has a curvature.

21. (canceled)

22. The vascular implant of claim 1 wherein the implant body and appendages are 3D printed of a material that cannot be machined.

23-24. (canceled)
